(19) United States
(12) Patent Application Publication
(10) Pub. No.: US 2014/0171385 A1
(43) Pub. Date: Jun. 19, 2014

(54) Materials for Short-Term Use in Mammals

(71) Applicant: ECD MEDICAL, San Clemente, CA (US)

(72) Inventors: Fred Burbank, Laguna Niguel, CA (US); Michael Jones, San Clemente, CA (US)

(73) Assignee: ECD MEDICAL, San Clemente, CA (US)

(21) Appl. No.: 14/105,463

(22) Filed: Dec. 13, 2013

Related U.S. Application Data
(60) Provisional application No. 61/737,272, filed on Dec. 14, 2012.

(51) Int. Cl. A61L 31/04; A61L 31/06; A61L 27/58; A61L 31/14; A61L 27/18; A61L 27/26

(52) U.S. Cl. CPC A61L 31/041; A61L 27/18; A61L 27/26; A61L 27/58; A61L 31/148; A61L 31/06
USPC 514/55; 525/450; 514/60; 523/105

(57) ABSTRACT

Biodegradable materials are formed by mixing together two or more materials which have different resorption times and different mechanical characteristics. Devices formed of these materials can be used in mammals in numerous medical and surgical applications, including those for which the mechanical properties of the device, left in vivo, much change over time.

### BACKGROUND

1. Field of Endeavor

The present invention relates to materials and processes useful in treatment of mammalian bodies, and more specifically to materials which degrade over time in vivo and processes of their use.

2. Brief Description of the Related Art

Resorbable materials have been part of the medical literature for quite a while. The most obvious example is resorbable “Gut” or Chromic Suture. A substantial body of work has been done to make a number of sutures resorbable; examples would include Vicryl, Poly-glycolic acid, and Polydioxanone which are synthesized to selectively hydrolyze and be resorbed by the body. Other examples of resorbable materials are staples or surgical clips used for ligation, poly-lactic acid screws used in orthopedic repairs, haemostatic materials such as starch, oxidized cellulose, or gel foam.

### SUMMARY

One of numerous aspects of the present inventions includes a bioresorbable device comprising a first subcomponent formed of a first bioresorbable material, and a second subcomponent formed of a second bioresorbable material, wherein the first bioresorbable material and the second bioresorbable material are mutually selected to degrade in vivo at at least two different rates.

### DETAILED DESCRIPTION OF EXEMPLARY EMBODIMENTS

In general terms, materials embodying principles of the present invention combine materials to provide for a structure which can have multiple resorption times frames depending on the end use of the product. The materials can be fabricated as a laminate, alloy, or composite providing the multiple resorption time frames.

For short term use, a single material, such as a sugar, starch, or other polysaccharide can be constructed utilizing a binder material such as polyethylene glycol, methyl cellulose, and/or hydroxy methyl cellulose, among many materials suitable for this use, to bind the starch particles together. This construction stays as an integral material until the binder absorbs sufficient water to swell and break apart. The starch is enzymatically degraded by the body in several days. These compressed starch materials are very strong under compressive loads but are not suitable for a tensile load.

In one use, a compressed starch is used to provide for the short term portion of the construction and an outer layer composed of poly-lactic acid, or polyglycolic acid, to provide a longer term construction that is suitable for tensile loading or as a snap fit piece. The starch would be degraded rather quickly, leaving the poly-lactic acid for longer term degradation.

An alternate embodiment uses a composite structure where a base polymer, such as polylactic acid, is used as a binder and sugar, starch, methyl cellulose, hydroxy methyl cellulose, or other polysaccharide is used as an aggregate. This construction would allow an initial rigid polymer to be introduced into the body and then, as the aggregate was dissolved into fluid, or enzymeatically degraded, the polylactic acid polymer would becomes substantially porous, quickly reducing its mechanical strength and allowing more rapid infusion of water for hydrolytic degradation. This construction could function short term in either tensile or compressive loading.

Another composite design would be a braided fiber such as resorbable suture that is placed into a beta glucan matrix. The beta glucan matrix provides a short term rigid piece and the fiber structure allows for tensile loading rather than just compressive loads.

Alternately, laminated designs, such as a chitosan construction over the fibers, could be used in place of the starch if a flexible member was needed.

Materials as described herein have numerous potential uses, including, but not limited to the following.

1) Vascular closure devices as described in a co-pending U.S. Provisional Application filed on even date herewith, entitled Vascular Closure Devices, bearing attorney reference number 099-002P, by Fred Burbank and Michael Jones.

3) Connectors for bypass grafts—these devices would be used to join the bypass graft (either artery or vein) to the host artery. Each end of the connector would be configured like the vascular closure device to bring the grafted artery into the main artery at an angle. The graft artery end would be placed over the guide and the fingers would slipped over the end of the artery, trapping it between the fingers and the guide.

4) In-situ tissue scaffolds—these devices are used as support structures and allow tissue healing to generate along a scaffold minimizing cosmetic, defects after a surgical procedure. The construction of these devices requires that the device be very porous to allow for infiltration during the healing process. These devices could be used for bone growth, nerve repair, soft tissue repair and potentially as a substrate for cartilage repair.

5) Temporary markers for biopsy sites—these devices are placed into biopsy sites and mark a lesion location after biopsy. During biopsies, such as breast biopsies, the target location is primarily identified by mammography as calcifications; once the biopsy is performed, the calcifications are mostly, if not completely, removed and thus not available to provide a location further intervention is required. This marker would be left behind to provide a visual or imageable location for subsequent therapy or surgery. The marker could be died with any of the FDA approved dyes/colorant used in sutures for use as a visual marker. The marker could optionally have surface porosity or surface bubbles which would make it identifiable from surrounding tissue on ultrasound. It could be labeled or contain a chelated gadolineum compound to be identifiable with MRI.

Materials as described herein can have numerous advantages over prior materials. A first advantage over the existing materials is that, instead of a single functional (i.e., degradation) time that each material provides, a blend of properties can be provided depending upon the need of the particular area in which the device made of the material is used. By mixing and blending the materials, numerous mechanical properties can be achieved, from short term rigidity to long term rigidity. Materials can be produced with immediate rigidity when dry for installation and then hydrate and soften, but have a fiber structure which will allows for tensile loading of the device.

With the blending or laminate construction, the longevity of the device in the body can vary. While the use of compressed starch is not new per se, it is easily degraded in the body in several days. If a device is needed in excess of several days, a longer term polymer, such as polylactic acid, can be added as an encapsulant or binder, using the starch or other polysaccharide as an aggregate similar to gravel in concrete. Although, in the case of the uses described herein, the aggregate is resorbed into the body, leaving a soft structure behind.

As used herein, the term “poly” means multiple repeating blocks of the monomer. For example, for a polyglycolic acid, the glycolide monomer is repeated numerous times. The molecular weight sufficient for a combination of mechanical and degradation properties is in the range of 10,000 to 20,000 Daltons. When the hydrolytic degradation produces chains with roughly 5,000 Daltons, the polymer is mobile within the body.

For a co-polymer such as poly lactide-co-glycolide, the chain of lactide molecules could be terminated by a single glycolide molecule, although in practice the ratio is more typically 90:10 (lactide:glycolide) to 20:80. Typically these become random copolymers with a wide range of inter-chain repeating units. This vastly decreases their longevity in the body as they do not form crystalline structures and hydrolytic degradation proceeds rapidly.

All materials listed herein would be representative and suitable for any genus to which each belongs. Their fabrication method may differ, but the fabricated unit should be functional regardless of the material.

### EXAMPLES

I. A compressed composition of starch with 20% (by weight percent) methyl cellulose is mixed as a binder. This material, when compressed at about 40,000 to 50,000 psi, becomes a useable solid material that has very good compressive strength, but little tensile strength.

II. A moldable composition includes a 65/35 copolymer of poly-lactic and poly-glycolic acid mixed with a short-term filler, such as starch or other polysaccharide, to accelerate its decomposition in vivo.

III. A first implantable sub-component is molded from a bioresorbable polymer such as 63/35 PLGA with resorption time of 6-8 weeks in vivo. A second, co-implanted sub-component is molded from a bioresorbable polymer such as 63/35 PLGA with resorption time of 6-8 weeks in vivo. A third co-implanted sub-component is molded from a bioresorbable polymer such as 63/35 PLGA with resorption time of 6-8 weeks in vivo. A fourth, co-implanted sub-component is made from a compressed starch with 20% by weight methyl cellulose as a binder. This fourth sub-component will be resorbed in the body in about 4-7 days.

II. A first implantable sub-component is compression molded from a bioresorbable, hemostatic starch with 20% by weight methyl cellulose as a binder. A second, co-implanted sub-component is molded from a bioresorbable polymer such as 63/35 PLGA with resorption time of 6-8 weeks in vivo. A third, co-implanted sub-component is molded from a bioresorbable polymer such as 63/35 PLGA with resorption time of 6-8 weeks in vivo. A fourth, co-implanted sub-component is made from a compressed starch with 20% by weight methyl cellulose as a binder. This fourth sub-component will be resorbed in the body in about 4-7 days.

III. A first implantable sub-component is compression molded from a bioresorbable, hemostatic chitosan with 20% by weight methyl cellulose as a binder. A second, co-implanted sub-component arm is molded from a bioresorbable polymer such as 63/35 PLGA with resorption time of 6-8 weeks in vivo. A third, co-implanted sub-component is molded from a bioresorbable polymer such as 63/35 PLGA with resorption time of 6-8 weeks in vivo. A fourth, co-implanted sub-component is made from a compressed starch with 20% by weight methyl cellulose as a binder. This fourth sub-component will be resorbed in the body in about 4-7 days.

IV. A first implantable sub-component is formed from a freeze dried bioresorbable, hemostatic chitosan. A second, co-implanted sub-component is molded from a bioresorbable polymer such as 63/35 PLGA with resorption time of 6-8 weeks in vivo. A third, co-implanted sub-component is molded from a bioresorbable polymer such as 63/35 PLGA with resorption time of 6-8 weeks in vivo. A fourth, co-implanted sub-component is made from a compressed starch with 20% by weight methyl cellulose as a binder. This fourth sub-component will be resorbed in the body in about 4-7 days.

V. A first implantable sub-component is molded from a bioresorbable polymer composite containing 20 to 50% by weight starch in a 63/35 PLGA with resorption time of 4-6 weeks in vivo. A second, co-implanted sub-component is molded from a bioresorbable polymer composite containing 20 to 50% by weight starch in a 63/35 PLGA. A third, co-implanted sub-component is molded from a bioresorbable polymer composite containing 20 to 50% by weight starch in a 63/35 PLGA. A fourth, co-implanted sub-component is made from a compressed starch with 20% by weight methyl cellulose as a binder. This fourth sub-component will be resorbed in the body in about 4-7 days.

VI. A first implantable sub-component is molded from a bioresorbable polymer composite containing 20 to 50% by weight chitosan in a 63/35 PLGA with resorption time of 4-6 weeks in vivo. A second, co-implanted sub-component is molded from a bioresorbable polymer composite containing 20 to 50% by weight starch in a 63/35 PLGA. A third, co-implanted sub-component is molded from a bioresorbable polymer composite containing 20 to 50% by weight starch in a 63/35 PLGA. A fourth, co-implanted sub-component is made from a compressed starch with 20% by weight methyl cellulose as a binder. This fourth sub-component will be resorbed in the body in about 4-7 days.

VII. A first implantable sub-component is formed from a freeze dried bioresorbable composite, of 20 to 50% by weight starch in chitosan. A second, co-implanted sub-component is molded from a bioresorbable polymer composite of 20 to 50% starch in 63/35 PLGA with resorption time of 4-6 weeks in vivo. A third, co-implanted sub-component is molded from a bioresorbable polymer composite of 20 to 50% starch in 63/35 PLGA with resorption time of 4-6 weeks in vivo. A fourth, co-implanted sub-component is made from a compressed starch with 20% by weight methyl cellulose as a binder. This fourth sub-component will be resorbed in the body in about 4-7 days.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

We claim:

1. A bioresorbable device comprising:
a first subcomponent formed of a first bioresorbable material; and
a second subcomponent formed of a second bioresorbable material;
wherein the first bioresorbable material and the second bioresorbable material are mutually selected to degrade in vivo at at least two different rates.

2. A bioresorbable device according to claim 1, wherein the first and second bioresorbable materials are each selected from the group consisting of:
63/35 PLGA;
a polymer composite containing 20 to 50% by weight starch in a 63/35 PLGA;
a bioresorbable, hemostatic chitosan with 20% by weight methyl cellulose as a binder;
a bioresorbable polymer composite containing 20 to 50% by weight starch in a 63/35 PLGA;
a bioresorbable polymer composite containing 20 to 50% by weight chitosan in a 63/35 PLGA;
a freeze dried bioresorbable, hemostatic chitosan;
a freeze dried bioresorbable composite of 20 to 50% by weight starch in chitosan;
a compressed starch with 20% by weight methyl cellulose as a binder; and
combinations thereof.

3. A bioresorbable device according to claim 1, wherein the first and second subcomponents each have a resorption time of 4-8 weeks in vivo.
